SEQUENCE LISTING (23 sequences)

SEQ ID NO: 1 - protein, 38 aa, Homo sapiens
DSHEKRHHGYRRKFHEKHHSHREFPFYGDYGSNYLYDN

SEQ ID NO: 2 - protein, 27 aa, Artificial Sequence
  Derivative of Histatin-1 peptide (SEQ ID NO 1)
RKFHEKHHSHREFPFYGDYGSNYLYDN

SEQ ID NO: 3 - protein, 25 aa, Artificial Sequence
  Derivative of Histatin-1 peptide (SEQ ID NO 1)
RKFHEKHHSHREFPFYGDYGSNYLY

SEQ ID NO: 4 - protein, 23 aa, Artificial Sequence
  Derivative of Histatin-1 peptide (SEQ ID NO 1)
RKFHEKHHSHREFPFYGDYGSNY

SEQ ID NO: 5 - protein, 21 aa, Artificial Sequence
  Derivative of Histatin-1 peptide (SEQ ID NO 1)
RKFHEKHHSHREFPFYGDYGS

SEQ ID NO: 6 - protein, 19 aa, Artificial Sequence
  Derivative of Histatin-1 peptide (SEQ ID NO 1)
RKFHEKHHSHREFPFYGDY

SEQ ID NO: 7 - protein, 17 aa, Artificial Sequence
  Derivative of Histatin-1 peptide (SEQ ID NO 1)
RKFHEKHHSHREFPFYG

SEQ ID NO: 8 - protein, 25 aa, Artificial Sequence
  Derivative of Histatin-1 peptide (SEQ ID NO 1)
FHEKHHSHREFPFYGDYGSNYLYDN

SEQ ID NO: 9 - protein, 23 aa, Artificial Sequence
  Derivative of Histatin-1 peptide (SEQ ID NO 1)
EKHHSHREFPFYGDYGSNYLYDN

SEQ ID NO: 10 - protein, 21 aa, Artificial Sequence
  Derivative of Histatin-1 peptide (SEQ ID NO 1)
HHSHREFPFYGDYGSNYLYDN

SEQ ID NO: 11 - protein, 19 aa, Artificial Sequence
  Derivative of Histatin-1 peptide (SEQ ID NO 1)
SHREFPFYGDYGSNYLYDN

SEQ ID NO: 12 - protein, 17 aa, Artificial Sequence
  Derivative of Histatin-1 peptide (SEQ ID NO 1)
REFPFYGDYGSNYLYDN

SEQ ID NO: 13 - protein, 15 aa, Artificial Sequence
  Derivative of Histatin-1 peptide (SEQ ID NO 1)
FPFYGDYGSNYLYDN

SEQ ID NO: 14 - protein, 21 aa, Artificial Sequence
  Derivative of Histatin-1 peptide (SEQ ID NO 1)
EKHHSHREFPFYGDYGSNYLY

SEQ ID NO: 15 - protein, 19 aa, Artificial Sequence
  Derivative of Histatin-1 peptide (SEQ ID NO 1)
HHSHREFPFYGDYGSNYLY

SEQ ID NO: 16 - protein, 17 aa, Artificial Sequence
  Derivative of Histatin-1 peptide (SEQ ID NO 1)
SHREFPFYGDYGSNYLY

SEQ ID NO: 17 - protein, 19 aa, Artificial Sequence
  Derivative of Histatin-1 peptide (SEQ ID NO 1)
EKHHSHREFPFYGDYGSNY

SEQ ID NO: 18 - protein, 17 aa, Artificial Sequence
  Derivative of Histatin-1 peptide (SEQ ID NO 1)
HHSHREFPFYGDYGSNY

SEQ ID NO: 19 - protein, 15 aa, Artificial Sequence
  Derivative of Histatin-1 peptide (SEQ ID NO 1)
SHREFPFYGDYGSNY

SEQ ID NO: 20 - protein, 17 aa, Artificial Sequence
  Derivative of Histatin-1 peptide (SEQ ID NO 1)
EKHHSHREFPFYGDYGS

SEQ ID NO: 21 - protein, 15 aa, Artificial Sequence
  Derivative of Histatin-1 peptide (SEQ ID NO 1)
HHSHREFPFYGDYGS

SEQ ID NO: 22 - protein, 13 aa, Artificial Sequence
  Derivative of Histatin-1 peptide (SEQ ID NO 1)
SHREFPFYGDYGS

SEQ ID NO: 23 - protein, 13 aa, Artificial Sequence
  Derivative of Histatin-1 peptide (SEQ ID NO 1)
SHREFPFYGDYGS

(19) United States
(12) Patent Application Publication
(10) Pub. No.: US 2023/0010110 A1
(43) Pub. Date: Jan. 12, 2023

(54) HISTATIN-1 FORMULATION FOR THE TREATMENT, REPAIR OR REGENERATION OF BONE TISSUE IN A SUBJECT

(71) Applicant: UNIVERSIDAD DE CHILE, Santiago (CL)

(72) Inventors: Vicente Armando TORRES GÓMEZ, Santiago (CL); Pedro Pablo TORRES SALAZAR, Santiago (CL); Mónica Andrea CÁCERES LLUCH, Santiago (CL)

(21) Appl. No.: 17/782,630

(22) PCT Filed: Dec. 7, 2020
(86) PCT No.: PCT/IB2020/061596

§ 371 Date: Jun. 3, 2022

Related U.S. Application Data
(60) Provisional application No. 62/944,713, filed on Dec. 6, 2019.

(51) Int. Cl. A61K 38/17; A61P 19/00; A61K 45/06; A61L 27/54; A61L 27/22; A61K 9/00

(52) U.S. Cl. CPC A61K 38/1729; A61P 19/00; A61K 45/06; A61L 27/54; A61L 27/22; A61K 9/0024; A61L 2430/02; A61L 2300/606; A61L 2300/252

(57) ABSTRACT

The present invention relates to a formulation or composition for the treatment, repair, formation or regeneration of bone tissue in a subject, comprising Histatin-1 or its derivatives. The present invention also relates to a biomaterial comprising Histatin-1 or its derivatives in a biocompatible material, and a method for the treatment, repair, formation or regeneration of bone tissues in a subject comprising administering to the subject a therapeutically effective amount of Histatin-1 or its derivatives.

### FIELD OF THE INVENTION

The present invention relates to a formulation or composition as a biomaterial for bone implant comprising Histatin-1 and its use for bone repair or regeneration in a subject.

### BACKGROUND OF THE INVENTION

Life expectancy is increasing worldwide and so is the risk of suffering either traumatic or chronic disease related damage of soft and hard tissues. Unfortunately, the efficiency of wound healing declines during aging, leading to the appearance of chronic ulcers, diabetic toe, periodontal disease, tooth loss and recurrent bone fractures, which are due to a decreased reparative capacity in injured tissues. The latter, together with an increased susceptibility of the elderly to incur with accidental trauma, contributes to deteriorate patient's quality of life, as well as having an important economic impact for the patients. In many respects, treatment of bone fractures is a major economic issue. For example, it is estimated that by 2017 among 6 countries of the European Union (Germany, Italy, France, Spain, Sweden and the United Kingdom), there were 2.7 million fragility fractures, which entailed a total cost of $37.5 billion EUR, and by 2030 this cost is expected to increase by 27%. In the case of the United States, the same upward trend is followed, and by 2025 more than 3 million fractures are expected, with an associated cost of $25.3 billion.

Besides aging-related trauma, the incidence of both low force and high force accident is increasing. For low force trauma, there are situations such as at-home fall and bone fragility, while for high force trauma, there is an important correlation with distractions associated with nowadays technologies, such as texting or phone calling, both of which increase by around 6-fold the incidence of traumatic accidents.

Problems with current therapies aiming to bone repair include imperfect union of fractures (nonunion, delayed union), systemic issues, such as septic traumatic fever, and indirect consequences, such as bone shortening, joint stiffness and avascular necrosis. Therapies traditionally employed for bone repair include fusion and internal fixation, which require the use of implants and filling materials that are effective in providing support for bone sealing and repositioning; however, a significant percentage of cases are associated with poor functional recovery, long lasting treatment and cost-effective handling. More recent therapies are being focused on tissue engineering approaches, by seeking osteoinductive and osteogenic capacities of different materials with biomedical applications. Hence, functionalized materials are gaining interest in the field of bone repair and orthopedics, with particular interest on bioactive materials with novel active molecules. Hence, the search for novel molecules that aid in bone reparation is an urgent matter.

In this scenario, and given the increasing rates of hard tissue trauma, it becomes challenging the development of efficient therapies to improve hard tissue repair, as it requires innovative research and the confluence of different disciplines to support it.

Since tissue engineering, multiple options have emerged that seek to return lost bone tissue, without relying on grafts, and always taking into account the normal process of bone repair. First, the usefulness of multiple materials (ceramic and polymeric) has been demonstrated, which, acting as scaffold, facilitate the migration and accommodation of bone and vascular cells in the damaged site; events that allow the much-needed formation of blood vessels (angiogenesis) and the synthesis of bone matrix (osteogenesis). Faced with this scenario, it is necessary to look for molecules of easy synthesis, which can be integrated into these scaffolds, in order to accelerate the bone repair process even more. WO90/15586 and WO99/17710 both describe biodegradable bone cements comprising bioactive molecules, such as a bone growth factor or antibiotics. The antibiotics disclosed therein (gentamycin, vancomycin, and aminoglycosides) however have a slow release profile, similar to that of the bone growth factor.

A variety of diseases and clinical disorders are treated by the administration of a pharmaceutically active peptide. One such example is prostate cancer, which is a sex hormone dependent cancer, and which can be treated by administration of a luteinizing hormone-releasing hormone (LHRH) analogue that disturbs the production of luteinizing hormone (LH), which regulates the synthesis of male hormones. In particular, to decrease LH production, peptide analogues of LHRH that act as superagonists of the luteinizing hormone releasing hormone receptor, such as leuprolide and goserelin, have been used.

In many instances, the therapeutic effectiveness of a pharmaceutically active peptide depends upon its continued presence in vivo over prolonged time periods. To achieve continuous delivery of the peptide in vivo, a sustained release or sustained delivery formulation is desirable, to avoid the need for repeated administrations.

There are currently FDA-approved products that use osteoinductive proteins to favor bone repair when added to matrices: Bone Morphogenetic Protein-2 (BMP-2) (Infuse®, Medtronic Inc); Platelet Derived Growth Factor (PDGF) (Augment®-GEM 21S®); Peptide P-15 (P-15) (I-Factor®) and Proteins derived from enamel matrix (EMD) (Emdogain®). Each one meets its clinical indication, however, they have strong limitations and disadvantages, for example: BMP-2 and PDGF are extremely labile proteins, added to the fact that both are associated with malignant transformation; BMP-2 has been reported to generate ectopic bone growth and exacerbated inflammatory responses, whereas EMD, which consists of a set of porcine proteins, is associated with undesired effects and contamination due to the extraction source (as for any xenograft). Finally, P-15, alike several products aforementioned, is mostly associated with cell adhesion, but leaving aside other relevant processes that are required for tissue repair, namely angiogenesis and osteogenesis.

As it could be noted, a simple molecule, protein or peptide (i.e. easily synthesized), capable of promoting several aspects required in hard tissue repair, such as angiogenesis and osteogenesis, lacking adverse effects, and being easily integrated into matrices, is not yet available. Hence, the identification of novel molecules that promote bone repair and that could be integrated into materials, represents an excellent opportunity.

The present invention relates to a formulation or biomaterial comprising Histatin-1 or a polynucleotide encoding it, and its use for bone repair or regeneration in a subject, on the basis of its osteoinductive and osteoconductive properties.

Histatin-1 peptide is recognized for its antimicrobial action, protective effect over dental structures, potent stimulating effect of oral epithelial migration and adhesion, as well as angiogenic, thus facilitating wound repair at the level of the oral mucosa, however, Histatin-1 therapeutic effect for bone repair or regeneration is something that until now has not been proposed nor reported.

### SUMMARY OF THE INVENTION

A formulation or biomaterial comprising Histatin-1 (or a polynucleotide encoding it) for bone repair or regeneration in a subject, including human, horse, dog, and cat. The formulation or biomaterial comprising Histatin-1 is osteoinductive and osteoconductive, as it promotes bone differentiation in damaged or ill tissues, which favors bone repair or regeneration in bone defects or musculoskeletal injuries, which might be associated or not with the following causes: traumatic (delayed union, non-union or pseudo-arthrosis, high energy traumatic injury), infectious (osteomyelitis, osteonecrosis), congenital (craniofacial cleft), surgical (bone resection), drug-related (drug related osteonecrosis of the jaw), age-related causes (fragility fractures related to osteoporosis), as well as other bone-related diseases such as arthrosis, spondylitis ankylosans, rickets, osteomalacia, osteogenesis imperfecta, marble bone disease (osteopetrosis), Paget disease of bone, and fibrous dysplasia.

### BRIEF DESCRIPTION OF THE DRAWINGS

FIG. 1 shows in vitro adhesion (A) and migration assays (B-D), using SAOS-2 osteosarcoma cells (A, B), MC3T3-E1 pre-osteoblasts (C) and mesenchymal cells derived from apical papilla (apical papilla mesenchymal cells, APMCs, (D)), which were grown in complete medium supplemented with bovine serum. It is seen that Histatin-1 favors both cell adhesion and migration. * p<0.05.

FIG. 2 shows an in vitro mineralization test (with alizarin red), using (A) SAOS-2 cells, (B) MC3T3-E1, (C) APMCs and (D) dental pulp mesenchymal cells, where the presence of Histatin-1 (7 days), increases the number of crystal nucleation sites. *p<0.05; ***p<0.001.

FIG. 3 shows the expression and activity of the enzyme Alkaline Phosphatase (ALP). In (A) SAOS-2 and (B) MC3T3-E1 cells were incubated in the absence or presence of 10 μM Histatin-1 for 24 h, and whole cell lysates were analyzed by Western blotting of ALP. Histatin-1 increases the protein levels of ALP. In (C) SAOS-2 cells were treated or not with 10 μM Histatin-1 for 24 and 48 h, and the activity of alkaline phosphatase (ALP) was measured by metabolization of the substrate p-nitrophenyl phosphate, following absorbance at 405 nm. Histatin-1 increases the activity of ALP. *p<0.05.

FIG. 4 shows the expression and localization of β-catenin in cells stimulated with Histatin-1. In (A) SAOS-2 cells were incubated in the absence or presence of 10 μM Histatin-1 for 24 h, and whole cell lysates were prepared for Western blot analysis of β-catenin. Histatin-1 increases the protein levels of β-catenin. In (B) SAOS-2 cells were incubated with Histatin-1 for 24 hours. Cells were then fixed, permeabilized and incubated with primary and secondary antibodies for subsequent immunofluorescence analysis of total and non-phosphorylated (transcriptionally active) β-catenin. Histatin-1 increases the nuclear localization of total and active β-catenin. *p<0.05; ***p <0.001.

FIG. 5 shows qPCR analysis for relative levels of osteoblastic differentiation markers. (A) MC3T3-E1 and (B) AMPCs cells were treated or not with 10 μM Histatin-1 for 7 days. Total RNA was extracted, for subsequent RT qPCR analysis of Runx2, osteocalcin, osteopontin and ALP. Histatin-1 increases the mRNA of different osteoblastic differentiation markers.

### DETAILED DESCRIPTION OF THE INVENTION

Bone repair or regeneration involves a series of key stages and steps that will favor and accelerate the correct recovery of tissues. Faced with a bone lesion, a clot develops, which will house immune, bone and vascular cells that will migrate to the tissue, where they will adhere. These cells clean the site, favor the formation of blood vessels (angiogenesis) for the arrival of nutrients, and the synthesis and remodeling of the lost bone matrix (osteogenesis) and mineral matrix.

Histatin-1 is a human salivary peptide of 38 amino acids responsible for maintenance of oral hemostasis, plays a role in forming acquired tooth pellicle and help in bonding of some metal ions. Proteolytic cleavage of Histatin-1 yields several derivative peptides, some of which maintain Histatin-1-original properties (FASEB J 2009; 23(11): 3928-3935; FASEB J 2009; 23(8): 2691-2701). Histatin-1 is recognized for its antimicrobial action and protection of dental structures, but also in recent years, has gained much interest for its potent stimulating effect of migration and oral epithelial adhesion, thus facilitating the repair of wounds at the level of the oral mucosa. However, its action as an aid in bone repair or regeneration is something that until now has not been proposed or disclosed.

Histatin-1 peptide has been identified as a novel pro-angiogenic factor, by stimulating endothelial cell migration and formation of endothelial structures in vitro and angiogenesis in vivo. Also, our recent studies indicate that Histatin-1 has a biological action on osteoblast lineage cells in culture, according to in vitro assays (J Periodontol 2019; 90(7): 766-774). Specifically, Histatin-1 restored both cell migration and viability in osteoblastic cells challenged with zoledronic acid, which is an antiresorptive drug (J Periodontol 2019; 90(7): 766-774). No other effect of Histatin-1 has been explored or suggested in osteoblastic cells and this is fundamental, as it will be described below.

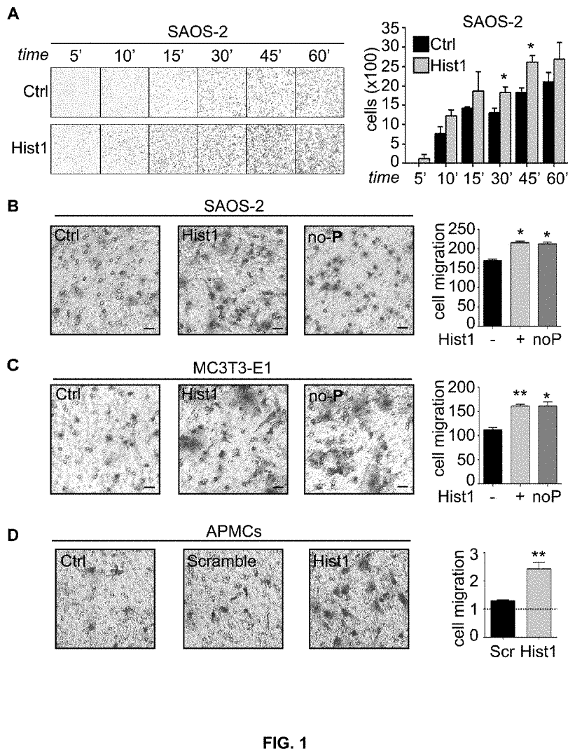

In the present invention is shown that in osteoblasts, pre-osteoblasts and mesenchymal cells, Histatin-1 promotes cell adhesion and migration, both events related with osteoconductivity (FIG. 1). The data shown in FIG. 1 prove the potential of Histatin-1 as an osteoconductive agent, which despite being relevant in bone regenerative medicine, is not sufficient to increase the efficiency of a given therapy. To improve the success of bone regenerative therapy, osteoinductivity, which refers to the ability of inducing bone cell differentiation and function, is mandatory and a key element in bone formation. Thus, the impact of Histatin-1 on bone cell differentiation and function (osteoinductivity) has not been previously reported, suggested nor proposed. This is highly relevant, because an osteoconductive agent is not necessarily osteoinductive.

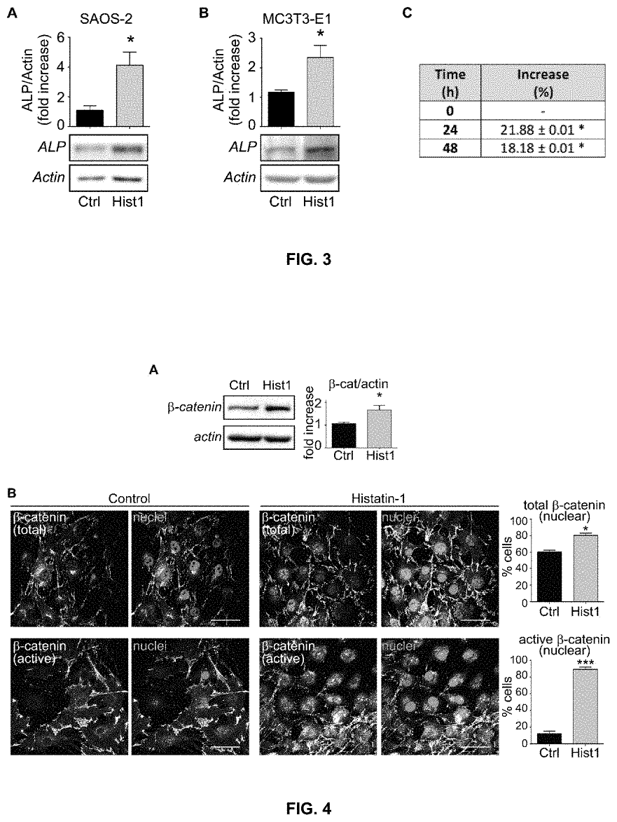

Preliminary results indicate that Histatin-1 can promote the expression and activity of Alkaline Phosphatase (ALP), in osteoblast-like cells (FIG. 3). Alkaline phosphatase is the enzyme necessary for the release of phosphate into the extracellular environment, which is necessary for the formation of crystal nucleation centers, an event that in turn is required for the synthesis of the mineral matrix of bone tissue (re-mineralization).

Another relevant characteristic of Histatin-1 is that it can promote the stabilization and activation of β-catenin in osteoblast-like cells, which is particularly relevant because β-catenin is associated with the induction of bone matrix synthesis (osteogenesis) (FIG. 4).

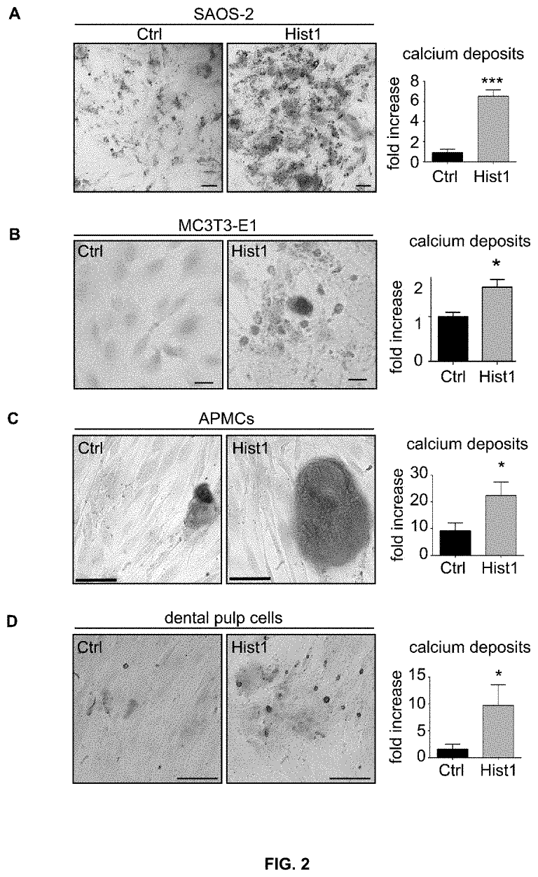

Even more relevant, via the alizarin red test it was demonstrated that osteoblastic lineage cells and mesenchymal stem cells derived from both dental pulp or the apical papilla of forming teeth exposed to Histatin-1 secrete a greater number of mineral nucleation centers; these nucleation centers suggest differentiation towards osteoblastic lineage (as shown in FIG. 2).

The stimulating effect of cell differentiation towards osteoblastic lineage of Histatin-1 was demonstrated using stem cells of apical papilla and dental pulp. Both types of stem cells can differentiate into bone cells under standardized conditions. The results indicate that Histatin-1 stimulates the formation of calcium deposits, indicating that Histatin-1 promotes the differentiation of stem cells towards bone lineage (FIG. 2).

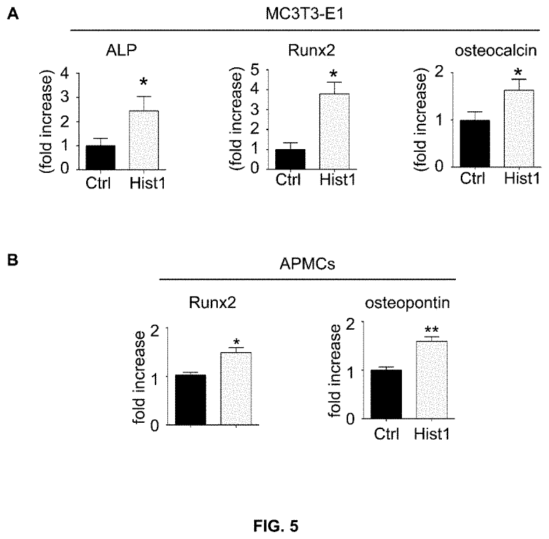

In addition, Histatin-1 induces an increase in the relative abundance of a number of key genes expressed during cell differentiation towards bone lineage (FIG. 5).

Thus, Histatin-1 new therapeutic use for bone repair or regeneration is based on:
pro-angiogenic effect in vitro and in vivo;
favors migration and adhesion in osteoblast and pre-osteoblast type cells;
protection against anti-migratory effects of cytotoxic drugs in bone cells;
promotes the expression of alkaline phosphatase (ALP);
promotes stabilization and activation of β-catenin;
promotes synthesis of bone matrix (osteogenesis) in culture;
induces bone differentiation genes;
induces synthesis of enzymes specific to functional bone cells.

The present invention relates to a formulation or biomaterial comprising Histatin-1 peptide (or a polynucleotide encoding it) for the treatment, repair, formation or regeneration of bone tissue in a subject, preferably mammalian subject, even more preferably human, horse, cat or dog.

The formulation or biomaterial comprising Histatin-1 peptide (or a polynucleotide encoding it) of the present invention allows an improved and accelerated bone repair or regeneration process, useful for clinical situations where the organism itself is exceeded, due to the extent of the lesion or ill tissue.

The amino acid sequence of human Histatin-1 peptide is disclosed in SEQ ID NO. 1. Thus, in some embodiments, the term “Histatin-1” includes a peptide having e.g. at least 85%, at least 90% or at least 99% sequence identity to SEQ ID NO. 1. Typically the peptide has this degree of sequence identity over at least 30 or at least 35 amino acid residues, or over the full length of the peptide.

The term “peptide” is used interchangeably herein to refer to polymers of amino acids of any length. The term also encompasses an amino acid polymer that has been modified (derivatives); for example, amidation, acetylation, disulfide bond formation, glycosylation, lipidation, phosphorylation, cyclization, or conjugation with a labeling component. More preferably, phosphorylation and/or cyclization.

A peptide has a certain percent “sequence identity” to another peptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489, 1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0

Therefore, the Histatin-1 peptide of the present invention comprises the amino acid sequence SEQ ID NO. 1, or its derivatives having at least 85%, at least 90% or at least 99% sequence identity to SEQ ID NO. 1. Also, cleaved forms of Histatin-1 peptide (including their derivatives, phosphorylated or non-phosphorylated forms and synthetic fragments) are suitable for the formulation of the present invention and their sequences are disclosed in SEQ ID NO. 2 to SEQ ID NO. 23. Therefore, the term “Histatin-1” is used interchangeably herein to refer to the Histatin-1 peptides of any of the SEQ ID NO. 1 to SEQ ID NO. 23, including its derivatives, and more preferably phosphorylated and/or cyclized peptides.

Furthermore, and in accordance with another alternative embodiment of the present invention, the disclosed Histatin-1 peptide or derivatives can also be modified with D-type amino acids and other chemical substances in order to increase the stability or half-life in the human body or to maintain an active structure.

Accordingly, in one aspect, the present invention is directed to a novel pharmaceutical composition or formulation for bone tissue regeneration treatment containing histatin-1 or derivatives (or a polynucleotide encoding it) as an active ingredient.

As used herein, the term “treatment” refers to any action for treating a bone disease, injury or any condition where bone regeneration is needed, by administering a pharmaceutical composition or implanting a biomaterial containing the peptide (or a polynucleotide encoding it), as an active ingredient to a subject in need of bone regeneration, in order to promote bone regeneration, repair or formation.

In the present invention, the formulation or pharmaceutical composition may be formulated as any of the group consisting of, but not limited to formulation for oral, buccal, mucosal, intranasal administration, for injection or for topical administration, and can be prepared into a suitable formulation using any well-known method in the state of art. (Joseph Price Remington, Remington's Pharmaceutical Science; 17th edition, Mack Publishing Company, Easton, Pa.).

In accordance with another alternative embodiment of the present invention, the formulation provides the local application of Histatin-1 peptide or its derivatives (or a polynucleotide encoding it) on a subject in need thereof, avoiding systemic effects and the use of supra-physiological concentrations.

In another aspect , the present invention provides a biomaterial, where said biomaterial is a biomaterial for bone graft that comprises Histatin-1 peptide or derivatives and a biocompatible material which can include: i) a synthetic polymer selected from the group consisting of polylactic glycolic acid, polylactic acid, cellulose, a poloxamer, polyurethanes, a polyhydroxyalkanoate-type polymer as poly(3-hydroxybutyrate-co-3-hydroxyvalerate) (PHBV), a polyester as polyglycolic acid, poly(ε-caprolactone) (PCL), an acrylamide copolymer as poly(N-isopropylacrylamide, and propylene glycol or a combination thereof; ii) a natural polymer selected from the group consisting of collagen, alginic acid, propylene glycol alginate, chondroitin sulfate and chitosan or a combination thereof; or iii) metal selected from Tantalum, Titanium, Iron, Magnesium and their alloys; or iv) natural or an artificial (chemically synthesizes) bone mineral, as organism-derived bone mineral powders and porous blocks, synthetic hydroxyapatite powders and porous blocks, tricalcium phosphate powders and porous blocks, monocalcium phosphate powders and porous blocks, bone graft materials containing silicon dioxide (silica), bone graft biomaterial and bone-packing graft materials consisting of a mixture of silica.

Said biomaterial can be used as any type of bone graft material and polymer scaffold used or reported in the state of art. Moreover, the peptide can be adsorbed onto the surface of the biocompatible material, embedded, covalently bonded, ionic bonded or dispersed into said material. In a further aspect, said peptide or derivatives can be encapsulated in or associated with particles, where said particles can be dispersed, coated on its surfaces, or embedded into the biocompatible material.

More particularly in the present invention, the biomaterial is preferably selected from the group consisting of chemically synthesizes bone minerals and synthetic polymers, but not limited thereto.

A biocompatible material is defined as a natural or synthetic material that is suitable for introduction into living tissue in an organism. It can also be defined as a material that shows biocompatibility, which is the ability of an implant material to function in vivo without eliciting detrimental local or systemic responses in the body. (Wright, T. M.; Maher, S. A. In Orthopaedic Basic Science: Foundations of Clinical Practice, 3rd ed).

In accordance with another aspect of the present invention, is provided the use of the peptide histatin-1 or derivatives (or a polynucleotide encoding it) or the pharmaceutical composition, for bone tissue regeneration treatment, including administering the peptide (or a polynucleotide encoding it), the pharmaceutical composition to a patient in need of treatment of bone tissue regeneration.

In another aspect, the present invention provides the use of biomaterial, for bone-tissue regeneration treatment, including grafting the biomaterial containing Histatin-1 or its derivatives to a patient in need of treatment of bone tissue regeneration.

When the formulation comprises a polynucleotide encoding Histatin-1 peptide or its derivatives, such polynucleotide is selected from commonly employed molecular biology constructs including DNA molecule, RNA molecule, aptamers, genetic construct, virus, adenovirus, adeno-associated virus, lentivirus, plasmid, artificial chromosome, natural vectors, and synthetic vectors, among others.

In another alternative embodiment of the present invention, the formulation provides a combination product of Histatin-1 peptide or derivatives with other usually prescribed active pharmaceutical ingredients, including small drug molecules from chemical synthesis, peptides, proteins, vaccines, lymphokines, enzymes, antibodies, hormones, hematopoietic factors, DNA derivatives, RNA derivatives, aptamers, stem cells, among others.

In addition, the formulation may be used alone or in combination or concomitantly with the biomaterial described on the present invention, or even with another bone graft (implant) material. Said bone graft material may include a bone mineral powder and a porous block thereof, a synthetic hydroxyapatite powder and a porous block thereof, a tricalcium phosphate powder and a porous block thereof, a monocalcium phosphate powder and a porous block thereof, and the like.

Additionally, the formulation can further comprise usually employed pharmaceutically acceptable excipients, including hydroxypropyl methyl cellulose, hydroxypropyl cellulose, carboxymethyl cellulose, ethyl cellulose and other cellulose derivatives, polycarbophil, carbopol, polyacrylate and polymethacrylate derivatives, polyglycolic acid, polylactic acid, poly(glycolic-co-lactic) acid, polycaprolactone, alginate, carrageenan, chitosan and other polysaccharide derivatives, chondroitin sulfate, hyaluronic acid and other glycosaminoglycan derivatives, polyarginine, polydioxanone, xanthan gum, guar gum, starch and starch derivatives, polyoxyethylene, polypropylene oxide, polyethylene glycol, D-alpha-tocopheryl polyethylene glycol succinate, among others. Additionally, the formulation can comprise plasticizing agents including tributyl citrate, acetyltributyl citrate, acetyltriethyl citrate, dibutyl sebacate, dibutyl phthalate and diethyl phthalate, glycerol, propylene glycol, and polyethylene glycol, among others.

In the present invention, the formulation may further contain at least one adjuvant selected but not limited ,from the group consisting of an excipient, a buffer, an antimicrobial antiseptic, a surfactant, an antioxidant, a tonicity adjuster, a preservative, a thickener and a viscosity modifier. Each ingredient can be selected from a raw material commonly used in the art and suitably modified and used within the range acceptable reported in the state of art.

In any embodiment of the present invention, the formulation or biomaterial comprising Histatin-1 or its derivatives (or a polynucleotide encoding it) is useful for the treatment, repair, or regeneration of bone tissue in a subject in need thereof. Here, such subject suffers from a bone defect or musculoskeletal injury, which might be associated or not with the following causes: traumatic (delayed union, non-union or pseudo-arthrosis, high energy traumatic injury), infectious (osteomyelitis, osteonecrosis), congenital (craniofacial cleft), surgical (bone resection), drug-related (drug related osteonecrosis of the jaw), age-related causes (fragility fractures related to osteoporosis) as well as other bone-related diseases such as arthrosis, spondylitis ankylosans, rickets, osteomalacia, osteogenesis imperfecta, marble bone disease (osteopetrosis), Paget disease of bone, and fibrous dysplasia.

In the present invention, the described biomaterial is preferably used for bone graft, but is not limited thereto. In another aspect, the present invention is aimed to the use of the peptide, pharmaceutical composition, or the biomaterial for bone tissue regeneration treatment.

In another aspect, the present invention is directed to a method for bone tissue regeneration treatment including administering (or grafting) the peptide, its derivatives (or a polynucleotide encoding it), the formulation or the biomaterial to a patient in need of treatment of bone tissue regeneration. Specifically, the subject to which the pharmaceutical composition is administered or to which the biomaterial can be grafted, implanted or attached , may be any animal including human, and may be, for example, an animal such as a dog, a cat, or a mouse.

The following examples are provided to illustrate certain embodiments of the invention and they are not intended to limit the invention in any way.

### EXAMPLES

### Example 1. Histatin-1 stimulates bone cell adhesion and migration, and in vitro mineralization

Histatin-1 was obtained by chemical synthesis, with the sequence SEQ ID NO. 1, corresponding to the full 38-amino acid peptide.

As shown in FIG. 1, Histatin 1 stimulates bone cell adhesion and migration. For this, (A) SAOS 2 cells were collected, resuspended in serum-free medium either containing or not 10 μM Histatin-1, and incubated for 30 min. Cells were then allowed to attach to fibronectin coated plates (2 μg/ml) for 5, 10, 15, 30, 45 and 60 min in serum-free medium, containing or not 10 μM Histatin-1. Cell adhesion was detected by crystal violet staining and counting under an optic microscope. Representative images are shown for each time point, in the absence or presence of Histatin 1. Graph shows quantification of 3 independent experiments performed in SAOS 2 cells (mean±s.e.; *p<0.05). (B, C and D) Cell migration was measured in Transwell chambers coated with 2 μg/ml fibronectin. Briefly, SAOS 2 (B), MC3T3 E1 cells (C) and APMCs (apical papilla mesenchymal cells, D) were allowed to migrate for 60 min in Transwell chambers, in the presence of 10 μM Histatin 1 or 10 μM of non-phosphorylated Histatin 1 (no P), 10 μM of a scramble sequence or vehicle control (H₂O). Cells that migrated were visualized by crystal violet staining. Representative images are shown, and graphs represent the averages of 3 independent experiments (mean±s.e.m; *p<0.05; **p<0.01).

The ability of Histatin-1 to stimulate the formation of calcium deposits was determined by the alizarin red staining test. For this, SAOS-2 cells, MC3T3-E1 preosteoblasts, stem cells from the apical papilla or dental pulp stem cells were grown in complete medium supplemented with bovine serum, in the absence or presence 10 μM Histatin-1 for 7 days. As shown in FIG. 2, (A) SAOS-2 cells, (B) MC3T3-E1, (C) APMCs and (D) dental pulp mesenchymal cells were incubated for 7 days in complete medium containing or not 10 μM Histatin-1, and then washed, fixed and stained with alizarin red solution. Cells were washed with PBS, fixed with 4% paraformaldehyde, and stained with Alizarin Red aqueous solution for 1 h. Finally, samples were washed with distilled water and subsequently with PBS. Calcium deposits were visualized in an optic microscope. Representative images are shown, and graphs represent the quantification of the amount of calcium deposits. Values are shown as the mean±s.e.m (*p<0.01; ***p<0.001).

Exposure of SAOS-2 and MC3T3-E1 cells to Histatin-1 increased staining of the culture with Alizarin Red, indicating that Histatin-1 stimulates the formation of calcium deposits in osteoblast lineage cells.

The same procedure was performed on stem cells of the apical papilla and dental pulp. In both cases, Histatin-1 stimulated the formation of calcium deposits, as observed by staining with Alizarin Red, indicating that Histatin-1 promotes the differentiation of stem cells towards bone lineage.

### Example 2. Histatin-1 stimulates the expression of alkaline phosphatase and β-catenin

Histatin-1 was obtained by chemical synthesis, with the sequence SEQ ID NO. 1, corresponding to the full 38-amino acid peptide.

By Western blot assay, the effect of Histatin-1 on the expression of two proteins involved in the osteogenesis process (alkaline phosphatase, β-catenin) was evaluated. For this, SAOS-2 or MC3T3-E1 cells were seeded on 60 mm plates, grown in complete medium supplemented with bovine serum for 24 h, washed with PBS and then incubated for 24 h with either serum-free medium or serum-free medium containing 10 μM Histatin-1. Cells were washed with PBS and then lysed in 0.2 mM HEPES (pH 7.4) buffer, containing 0.1% SDS, and protease and phosphatase inhibitors cocktail. Total protein extracts were separated by SDS-polyacrylamide gel electrophoresis and transferred to nitrocellulose membranes. Blots were blocked with 5% milk in TBS-Tween20 (0.1%) and incubated with antibodies against alkaline phosphatase, β-catenin, and actin. Bound first antibodies were detected with HRP-conjugated second antibodies and detected with the EZ-ECL system.

As shown in FIG. 3, (A) SAOS 2 and (B) MC3T3 E1 cells were incubated in the absence or presence of 10 μM Histatin 1 for 24 h, and whole cell lysates were prepared for Western blot analysis of alkaline phosphatase (ALP) and actin. Representative Western blot images are shown, and relative levels of ALP were quantified by scanning densitometry and normalized to actin. Graphs represent the average from 3 independent experiments (mean±s.e.m.; *p<0.05). (C) SAOS 2 cells were treated or not with 10 μM Histatin 1 for 24 and 48 h, and the activity of alkaline phosphatase (ALP) was measured by metabolization of the substrate p-nitrophenyl phosphate, following absorbance at 405 nm. Data were averaged from 3 independent experiments and shown as residual activity (% increase with respect to the control condition; mean±s.e.m; *p<0.05).

As shown in FIG. 4, (A) SAOS 2 cells were treated or not with 10 μM Histatin 1 for 24 h, and whole cell lysates were prepared for Western blot analysis of β catenin and actin. Representative Western blot images are shown, and relative levels of β catenin were quantified by scanning densitometry and normalized to actin. Graphs represent the average from 3 independent experiments (mean±s.e.m.; *p<0.05). (B) SAOS 2 cells were treated or not with 10 μM Histatin 1 for 24 h, then fixed, permeabilized and analyzed incubated with primary and secondary antibodies for subsequent immunofluorescence analysis of total and non-phosphorylated (transcriptionally active) β-catenin. Left panels show representative confocal microscopy images of total and active β-catenin, whereas nuclei staining with DAPI was used as reference. Upper and lower right graphs show the percentage of cells with nuclear accumulation of total and transcriptionally active β catenin, respectively, and obtained by averaging 3 independent experiments (mean±s.e.m.; *p<0.05; ***p<0.001).

It was observed that incubation of SAOS-2 cells with Histatin-1 increased the production of this enzyme. On the other hand, the evaluation of the level of expression of the β-catenin protein indicated that Histatin-1 increases the expression of the protein. Alternatively, SAOS-2 cells grown on coverslips were fixed, permeabilized and analyzed for total and active β-catenin nuclear localization.

### Example 3. Histatin-1 stimulates the expression of bone differentiation genes

Histatin-1 was obtained by chemical synthesis, with the sequence SEQ ID NO. 1, corresponding to the full 38-amino acid peptide.

To evaluate the differentiation towards bone lineage in the presence of Histatin-1, the expression of the osteocalcin, osteopontin, Runx2 and ALP gene were determined, as markers of differentiation, by means of a qPCR assay for mRNA content analysis. For this, osteoblasts-like cells and stem cells from the apical papilla were grown in complete medium supplemented with bovine serum, in the absence or presence of 10 μM Histatin-1 for 24 h. Total RNA was extracted with TRIZOL (Invitrogen, Life Technologies), following the manufacturer's instructions. For cDNA synthesis, samples were treated with RNase-Free DNase kit (#M6101 Promega), quantified and purity was verified for subsequent reverse transcription, using the cDNA Reverse Transcription Kit (Applied Biosystems). Alkaline phosphatase, osteocalcin, osteopontin, Runx2 and GAPDH were quantified by qRT-PCR (Applied Biosystems, ΔΔCt method).

As shown in FIG. 5, (A) MC3T3 E1 and (B) APMCs cells were treated or not with 10 μM Histatin 1 for 7 days. Total RNA was extracted, for subsequent RT qPCR analysis of Runx2, osteocalcin, osteopontin and ALP. Relative abundance of all screened mRNAs was normalized to GAPDH, by using the ΔΔCT method. Graphs show data obtained by averaging 3 independent experiments (mean±s.e.m.; *p<0.05; **p<0.01).

The results indicate that treatment with Histatin-1 induces an increase in the relative abundance of mRNA for key genes expressed during bone cell differentiation.

1. A formulation for the treatment, repair, formation, or regeneration of bone tissue in a subject, wherein said formulation comprises Histatin-1 or its derivatives.

2. A formulation for the treatment, repair, formation, or regeneration of bone tissue in a subject, wherein said formulation comprises a polynucleotide encoding Histatin-1 or its derivatives.

3. The formulation according to claim 1 or 2, wherein said Histatin-1 or its derivatives comprises an amino acid sequence having at least 85%, at least 90% or at least 99% sequence identity to any of SEQ ID NO. 1 to SEQ ID NO. 23.

4. The formulation according to claim 1, 2 or 3, wherein said derivatives comprises a modification selected from disulfide bond formation, glycosylation, lipidation, phosphorylation, cyclization, and conjugation with a labeling component.

5. The formulation according to claim 1 or 2, wherein said formulation further comprises an additional active pharmaceutical ingredient.

6. A biomaterial for the treatment, repair, formation, or regeneration of bone tissue in a subject, wherein said material comprises Histatin-1 or its derivatives in a biocompatible material.

7. The biomaterial according to claim 6, wherein said Histatin-1 or its derivatives comprises an amino acid sequence having at least 85%, at least 90% or at least 99% sequence identity to any of SEQ ID NO. 1 to SEQ ID NO. 2.

8. The biomaterial according to claim 6 or 7, wherein said derivatives comprises a modification selected from disulfide bond formation, glycosylation, lipidation, phosphorylation, cyclization, and conjugation with a labeling component.

9. The biocompatible material according to claim 6, wherein said biocompatible material is selected from bone mineral, metals, natural polymers, and synthetic polymers.

10. A biomaterial according to claim 8 having the Histatin-1 or its derivatives dispersed, coated on its surfaces, or embedded into the biocompatible material.

11. A biocompatible material according to claim 7, wherein the biocompatible material is selected from the group consisting of: organism-derived bone mineral powders, organism-derived bone mineral porous blocks, synthetic hydroxyapatite powders and porous blocks, tricalcium phosphate powders and porous blocks, monocalcium phosphate powders and porous blocks, bone graft materials containing silicon dioxide (silica), bone graft biomaterial ,bone-packing graft materials consisting of a mixture of silica and polymer, fine particles and porous scaffolds containing biocompatible polymers, titanium, and three-dimensional porous scaffolds; collagent, alginic acid, polylactic acid, polyglycolic acid, poly(ε-caprolactone) (PCL), poly(3-hydroxybutyrate-co-3-hydroxyvalerate) (PHBV), cellulose, poly(N-isopropylacrylamide), gelatin, propylene glycol, chondroitin sulfate and chitosan; polylactic glycolic acid, poloxamer and propylene glycol.

12. The formulation of claim 5, wherein said additional active pharmaceutical ingredient is selected from small drug molecules, peptides, proteins, vaccines, lymphokines, enzymes, antibodies, stem cells, secretomes, hormones, hematopoietic factors, DNA derivatives, RNA derivatives and aptamers.

13. The formulation according to claim 1, 2 or 5, wherein said formulation further comprises a pharmaceutically acceptable excipient.

14. The formulation of claim 13, wherein said pharmaceutically acceptable excipient is selected from hydroxypropyl methyl cellulose, hydroxypropyl cellulose, carboxymethyl cellulose, ethyl cellulose and other cellulose derivatives, polycarbophil, carbopol, polyacrylate and polymethacrylate derivatives, polyglycolic acid, polylactic acid, poly(glycolic-co-lactic) acid, polycaprolactone, alginate, carrageenan, chitosan and other polysaccharide derivatives, chondroitin sulfate, hyaluronic acid and other glycosaminoglycan derivatives, polyarginine, polydioxanone, xanthan gum, guar gum, starch and starch derivatives, polyoxyethylene, polypropylene oxide, polyethylene glycol, D-alpha-tocopheryl polyethylene glycol succinate, tributyl citrate, acetyltributyl citrate, acetyltriethyl citrate, dibutyl sebacate, dibutyl phthalate and diethyl phthalate, glycerol, propylene glycol, and polyethylene glycol.

15. The biomaterial according to claim 6, wherein said biomaterial is applied, attached, grafted, or implanted to a subject in need of bone treatment, repair, formation, or regeneration.

16. The formulation according to claim 1 or 2, wherein said formulation is administered systemically, transmucosally or locally to the subject.

17. The formulation according to claim 1, 2 or 15, wherein said subject is a mammalian subject selected from human, horse, cat, and dog.

18. The formulation according to claim 1, 2 or 15, wherein said subject suffers from bone defect, bone fracture, bone resection, craniofacial cleft, musculoskeletal injury, arthrosis, pseudo-arthrosis, osteoporosis, osteomyelitis, osteonecrosis, spondylitis ankylosans, rickets, osteomalacia, osteogenesis imperfecta, marble bone disease (osteopetrosis), Paget disease of bone, and fibrous dysplasia.

19. The formulation of claim 2, wherein said polynucleotide is selected from DNA molecule, RNA molecule, aptamers, genetic construct, virus, adenovirus, adeno-associated virus, lentivirus, plasmid, artificial chromosome, natural vectors, and synthetic vectors, among others.

20. A method for the treatment, repair, formation, or regeneration of bone tissues in a subject, wherein the method comprises administering to the subject a therapeutically effective amount of Histatin-1 or its derivatives.

21. A method for the treatment, repair, formation, or regeneration of bone tissues in a subject, wherein the method comprises administering to the subject a therapeutically effective amount of a polynucleotide encoding Histatin-1 or its derivatives.

22. The method according to claim 20 or 21, wherein said Histatin-1 or its derivatives comprises an amino acid sequence having at least 85%, at least 90% or at least 99% sequence identity to any of SEQ ID NO. 1 to SEQ ID NO. 23.

23. The method according to claim 20, 21 or 22, wherein said derivative comprises a modification selected from disulfide bond formation, glycosylation, lipidation, phosphorylation, cyclization, and conjugation with a labeling component.

24. The method according to claim 20 or 21, wherein said formulation further comprises an additional active pharmaceutical ingredient.

25. The method according to claim 24, wherein said additional active pharmaceutical ingredient is selected from small drug molecules, peptides, proteins, vaccines, lymphokines, enzymes, antibodies, stem cells, secretome, hormones, hematopoietic factors, DNA derivatives, RNA derivatives and aptamers.

26. The method according to claim 20, 21 or 24, wherein said formulation further comprises a pharmaceutically acceptable excipient.

27. The method according to claim 26, wherein said pharmaceutically acceptable excipient is selected from hydroxypropyl methyl cellulose, hydroxypropyl cellulose, carboxymethyl cellulose, ethyl cellulose and other cellulose derivatives, polycarbophil, carbopol, polyacrylate and polymethacrylate derivatives, polyglycolic acid, polylactic acid, poly(glycolic-co-lactic) acid, polycaprolactone, alginate, carrageenan, chitosan and other polysaccharide derivatives, chondroitin sulfate, hyaluronic acid and other glycosaminoglycan derivatives, polyarginine, polydioxanone, xanthan gum, guar gum, starch and starch derivatives, polyoxyethylene, polypropylene oxide, polyethylene glycol, D-alpha-tocopheryl polyethylene glycol succinate, tributyl citrate, acetyltributyl citrate, acetyltriethyl citrate, dibutyl sebacate, dibutyl phthalate and diethyl phthalate, glycerol, propylene glycol, and polyethylene glycol.

28. The method according to claim 20 or 21, wherein said formulation is administered systemically or locally to the subject.

29. The method according to claim 20 or 21, wherein said subject is a mammalian subject selected from human, horse, cat, and dog.

30. The method according to claim 20 or 21, wherein said subject suffers from bone defect, bone fracture, bone resection, craniofacial cleft, musculoskeletal injury, arthrosis, pseudo-arthrosis, osteoporosis, osteomyelitis, osteonecrosis, spondylitis ankylosans, rickets, osteomalacia, osteogenesis imperfecta, marble bone disease (osteopetrosis), Paget disease of bone, or fibrous dysplasia.

31. The method according to claim 21, wherein said polynucleotide is selected from DNA molecule, RNA molecule, aptamers, genetic construct, virus, adenovirus, adeno-associated virus, lentivirus, plasmid, artificial chromosome, natural vectors, and synthetic vectors, among others.

32. A method for the treatment, repair , formation or regeneration of bone tissues in a subject, wherein the method comprises the application, grafting, attaching or implantation of a biomaterial, that comprises Histatin-1 or its derivatives in a biocompatible material, in a subject in need of bone repair, formation or regeneration.

33. The method according to claim 32 wherein said method further comprises administering the biomaterial in combination with a bone graft implant.

34. The method according to claim 32 wherein said subject is a mammalian subject selected from human, horse, cat, and dog.

35. The method according to claim 34 wherein said subject suffers from bone defect, bone fracture, bone resection, craniofacial cleft, musculoskeletal injury, arthrosis, pseudo-arthrosis, osteoporosis, osteomyelitis, osteonecrosis, spondylitis ankylosans, rickets, osteomalacia, osteogenesis imperfecta, marble bone disease (osteopetrosis), Paget disease of bone, or fibrous dysplasia.
